(19) United States
(12) Patent Application Publication
(10) Pub. No.: US 2022/0031877 A1
(43) Pub. Date: Feb. 3, 2022

(54) CLEANING AND STERILIZING DEVICE

(71) Applicant: GOOTEN INNOLIFE CORPORATION, Taichung City (TW)

(72) Inventor: YI-WEI LIAO, TAICHUNG CITY (TW)

(21) Appl. No.: 16/983,783

(22) Filed: Aug. 3, 2020

(51) Int. Cl. A61L 2/10; B08B 3/12

(52) U.S. Cl. CPC A61L 2/10; A61L 2202/121; A61L 2202/11; B08B 3/12

(57) ABSTRACT

A cleaning and sterilizing device includes an ultrasonic cleaner, a top cover and an ultraviolet lamp. The ultrasonic cleaner has a cleaning tank for performing ultrasonic cleaning process to objects. The top cover is disposed on the opening of the cleaning tank and removable at any time. The ultraviolet lamp is installed on the bottom surface of the top cover and directly faces the cleaning tank for providing sterilization effect to the object in the cleaning tank. Therefore, the cleaning and sterilizing device of the present invention is optional to clean the object by ultrasonic cleaning or sterilize the object by irradiating it by the ultraviolet lamp, thereby attaining the objective of thorough cleaning and sterilization. Besides, the top cover with the detachable design brings convenience for adding or pouring the water in the cleaning tank, thereby increasing the usage convenience.

### BACKGROUND OF THE INVENTION

### 1. Field of the Invention

The present invention relates to cleaning and sterilizing devices and more particularly, to a cleaning and sterilizing device with both functions of ultrasonic cleaning and ultraviolet sterilizing.

### 2. Description of the Related Art

The theorem of the cleaning device is using ultrasound to vibrate water, so that countless tiny vacuum bubbles are produced in the water. When the vacuum bubbles are burst under pressure, the energy stored therein is released. The aforementioned energy can impact the surface, corner or gap of an object to make the grease or dirt attached at these places gradually come off, thereby attaining the cleaning effect. However, the current cleaning devices lack the sterilizing function, that is liable to cause the problem of bacterial growth to some objects requiring thorough cleaning and sterilization, such as dentures.

### SUMMARY OF THE INVENTION

It is a primary objective of the present invention to provide a cleaning and sterilizing device which has both functions of ultrasonic cleaning and ultraviolet sterilizing.

To attain the above primary objective, the present invention provides a cleaning and sterilizing device which includes an ultrasonic cleaner, a top cover and an ultraviolet lamp. The ultrasonic cleaner has a cleaning tank for performing ultrasonic cleaning process to objects. The top cover detachably covers the opening of the cleaning tank. When the top cover is detached, the user can add water into the cleaning tank or pour off the water in the cleaning tank. The ultraviolet lamp is disposed on the bottom surface of the top cover and directly faces the cleaning tank for sterilizing the object in the cleaning tank.

It can be known from the above description that the cleaning and sterilizing device of the present invention is optional to clean the object by ultrasonic cleaning or sterilize the object by irradiating it by the ultraviolet lamp, thereby attaining the objective of thorough cleaning and sterilization. Besides, the top cover with the detachable design brings convenience for adding or pouring the water in the cleaning tank, thereby increasing the usage convenience.

Preferably, the top cover has a covering plate and a handle; the covering plate has an opening penetrating through top and bottom surfaces of the covering plate, and the top surface of the covering plate has an accommodating trough communicating with the opening of the covering plate and an engaging portion provided around the opening of the covering plate; the handle is disposed on the top surface of the covering plate and covers the accommodating trough; the ultraviolet lamp is disposed on the engaging portion of the covering plate and exposed on the bottom surface of the covering plate through the opening of the covering plate. The afore-described technical features enable the user to hold the handle to move it together with the covering plate and the ultraviolet lamp for placing them on the opening of the cleaning tank or removing them from the opening of the cleaning tank, thereby increasing the usage convenience.

Preferably, the ultrasonic cleaner has two first electrical contacts arranged abreast; the covering plate is provided on one side thereof with two through holes arranged abreast and communicating with the accommodating trough; two conductive wires located in the accommodating trough are connected to two ends of the ultraviolet lamp respectively; an end of each of the conductive wires is connected to a second electrical contact; the second electrical contacts are located at the through holes. Through the afore-described technical features, the two second electrical contacts of the ultraviolet lamp can be in contact with the first electrical contacts of the ultrasonic cleaner when the cleaning tank is covered by the top cover, so that the electrical conduction of the ultraviolet lamp is achieved. After that, the ultraviolet lamp can be controlled to generate ultraviolet when the power is turned on.

Preferably, a reflector is disposed above the ultraviolet lamp. By the reflector, the ultraviolet generated by the ultraviolet lamp can be relatively more concentrated to irradiate on the surface of the object.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a perspective view of a cleaning and sterilizing device of the present invention.

FIG. 2 is a partially exploded view of the cleaning and sterilizing device of the present invention.

FIG. 3 is an exploded perspective view of a part of the cleaning and sterilizing device of the present invention.

FIG. 4 is a partially cut-off top view of the cleaning and sterilizing device of the present invention when a handle and a reflector is removed.

FIG. 5 is a partially cut-off side view of the cleaning and sterilizing device of the present invention.

### DETAILED DESCRIPTION OF THE INVENTION

First of all, it is to be mentioned that throughout this specification, including the following embodiment and claims, the directional terms are all based on the direction shown in the figures. Besides, same reference numerals used in the following embodiment and the appendix drawings designate same or similar elements or the structural features thereof.

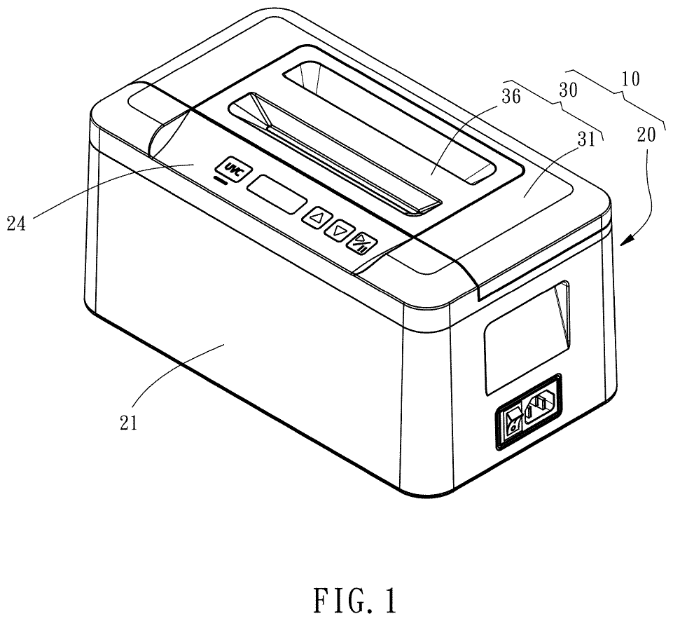

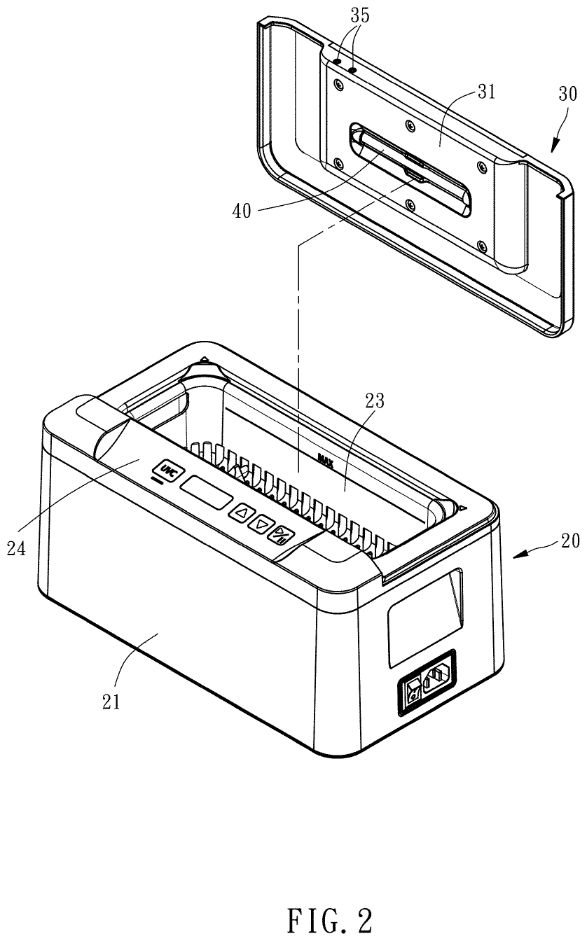

Referring to FIGS. 1-3, a cleaning and sterilizing device 10 of the present invention includes an ultrasonic cleaner 20, a top cover 30 and an ultraviolet lamp 40.

The ultrasonic cleaner 20 has a body 21 having a cleaning tank 22. A cleaning basket 23 is disposed in the cleaning tank 22 for accommodating objects. A control panel cover 24 is disposed on the top edge of the body 21 and surrounds the cleaning tank 22. A circuit board 26 is disposed in the control panel cover 24 and provided with two first electrical contacts 27 arranged abreast. One of the first electrical contacts 27 is the positive electrode and the other is the negative electrode. The two first electrical contacts 27 are inserted in two first through holes 25 of the control panel cover 24. Besides, the body 21 is provided with at least one vibration element below the cleaning tank 22. The vibration element, which belongs to the prior art and is not shown in the figures, can be controlled by the circuit board 26 to generate ultrasonic frequency oscillation.

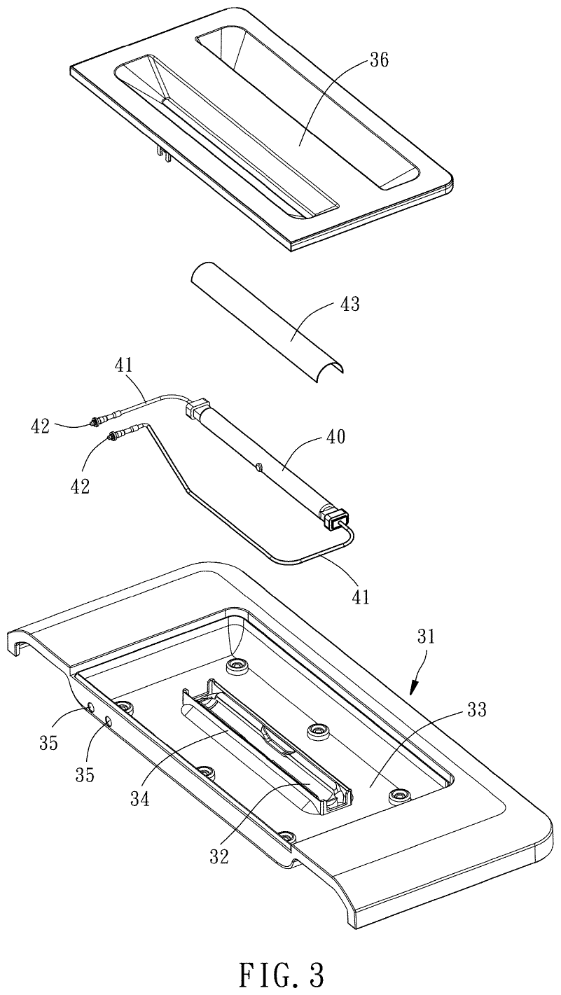

As shown in FIG. 3, the top cover 30 has a covering plate 31 and a handle 36. The covering plate 31 is provided at the center thereof with an elongated opening 32 penetrating through top and bottom surfaces of the covering plate. The top surface of the covering plate 31 has an accommodating trough 33 communicating with the opening 32 and an engaging portion 34 provided around the opening 32. The covering plate 31 is provided on one side thereof with two second through holes 35 arranged abreast and communicating with the accommodating trough 33. The handle 36 is disposed on the top surface of the covering plate 31 in a screwing manner and covers the accommodating trough 33. In this way, on one hand, the user can hold the handle 36 to make the top cover 30 cover the opening of the cleaning tank 22. At this time, the control panel cover 24 receives the bottom surface of the covering plate 31 for support. On the other hand, the user can hold the handle 36 to detach the top cover 30 from the opening of the cleaning tank 22 to conveniently add water into the cleaning tank 22 or pour water from it.

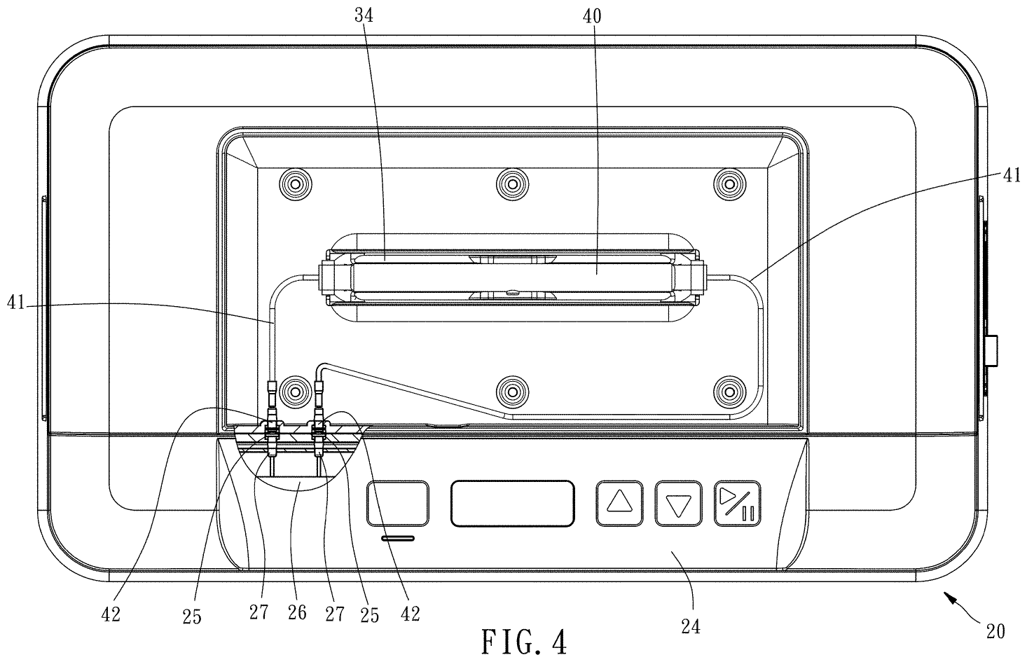

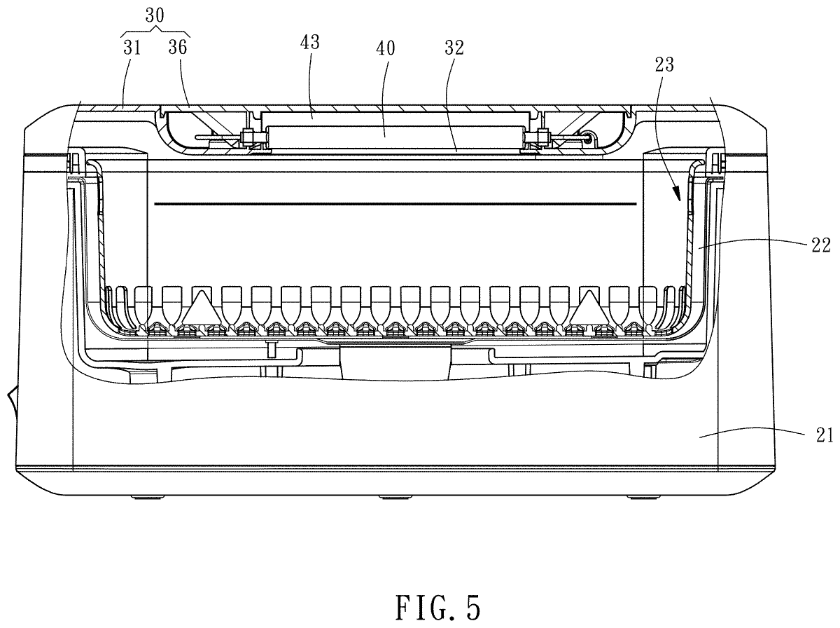

The ultraviolet lamp 40 is disposed on the engaging portion 34 of the covering plate 31 and exposed on the bottom surface of the covering plate 31 through the opening 32. Two conductive wires 41 are connected to two ends of the ultraviolet lamp 40 respectively. The two conductive wires 41 are both located in the accommodating trough 33, and an end of each of the conductive wires 41 is connected to a second electrical contact 42. That means there are two second electrical contacts 42, one of which is the positive electrode and the other is the negative electrode. The two second electrical contacts 42 are inserted in the two second through holes 35 of the covering plate 31. In this way, as shown in FIGS. 4 and 5, when the opening of the cleaning tank 22 is covered by the top cover 30, the ultraviolet lamp 40 directly faces the cleaning tank 22, and the two second electrical contacts 42 of the ultraviolet lamp 40 contact the two first electrical contacts 27 of the ultrasonic cleaner 20, so that the electrical conduction of the ultraviolet lamp 40 is achieved. After that, when the power is turned on, the ultraviolet lamp 40 can be controlled to generate ultraviolet to sterilize the object located in the cleaning tank 22. In addition, an arc reflector 43 may be further disposed above the ultraviolet lamp 40, as shown in FIG. 3. Through the reflector 43, the ultraviolet generated by the ultraviolet lamp 40 can be relatively more concentrated to irradiate on the surface of the object.

It can be known from the above description that the cleaning and sterilizing device 10 of the present invention can clean the object by ultrasonic cleaning or sterilize the object by irradiating it by the ultraviolet lamp, thereby attaining the objective of thorough cleaning and sterilization. Besides, by the detachable design of the top cover 30, the ultraviolet lamp 40 can be used right after the top cover 30 covers the cleaning tank 22, and it is convenient to add water or pour water after the top cover 30 is detached. Therefore, the ultrasonic cleaner 20 and the ultraviolet lamp 40 are quite convenient to be used in coordination.

What is claimed is:

1. A cleaning and sterilizing device comprising:
an ultrasonic cleaner having a cleaning tank;
a top cover detachably covering an opening of the cleaning tank; and
an ultraviolet lamp disposed on a bottom surface of the top cover and directly facing the cleaning tank.

2. The cleaning and sterilizing device as claimed in claim 1, wherein the top cover has a covering plate and a handle; the covering plate has an opening penetrating through top and bottom surfaces of the covering plate, and the top surface of the covering plate has an accommodating trough communicating with the opening of the covering plate and an engaging portion provided around the opening of the covering plate; the handle is disposed on the top surface of the covering plate and covers the accommodating trough; the ultraviolet lamp is disposed on the engaging portion of the covering plate and exposed on the bottom surface of the covering plate through the opening of the covering plate.

3. The cleaning and sterilizing device as claimed in claim 2, wherein the ultrasonic cleaner has two first electrical contacts arranged abreast; the covering plate is provided on one side thereof with two second through holes arranged abreast and communicating with the accommodating trough; two conductive wires located in the accommodating trough are connected to two ends of the ultraviolet lamp respectively; an end of each of the conductive wires is connected to a second electrical contact; the second electrical contacts are inserted in the second through holes and electrically contact the first electrical contacts of the ultrasonic cleaner.

4. The cleaning and sterilizing device as claimed in claim 3, wherein the ultrasonic cleaner is provided on a top edge thereof with a control panel cover surrounding the cleaning tank; the control panel cover receives the bottom surface of the covering plate and has two first through holes; a circuit board is disposed in the control panel cover and provided with the first electrical contacts; the first electrical contacts are inserted in the first through holes.

5. The cleaning and sterilizing device as claimed in claim 1, wherein a reflector is disposed above the ultraviolet lamp.
